(19) United States
(12) Patent Application Publication
(10) Pub. No.: US 2015/0302158 A1
(43) Pub. Date: Oct. 22, 2015

(54) VIDEO-BASED PULSE MEASUREMENT

(71) Applicant: Microsoft Corporation, Redmond, WA (US)

(72) Inventors: Daniel Scott Morris, Bellevue, WA (US); Siddharth Khullar, Malden, MA (US); Neel Suresh Joshi, Seattle, WA (US); Timothy Scott Saponas, Woodinville, WA (US); Desney S. Tan, Kirkland, WA (US)

(73) Assignee: Microsoft Corporation, Redmond, WA (US)

(21) Appl. No.: 14/257,671

(22) Filed: Apr. 21, 2014

(51) Int. Cl. G06F 19/00; A61B 5/00; A61B 5/024

(52) U.S. Cl. CPC G06F 19/3406; A61B 5/02427; A61B 5/7214; A61B 5/02405

(57) ABSTRACT

Aspects of the subject disclosure are directed towards a video-based pulse/heart rate system that may use motion data to reduce or eliminate the effects of motion on pulse detection. Signal quality may be computed from (e.g., transformed) video signal data, such as by providing video signal feature data to a trained classifier that provides a measure of the quality of pulse information in each signal. Based upon the signal quality data, corresponding waveforms may be processed to select one for extracting pulse information therefrom. Heart rate data may be computed from the extracted pulse information, which may be smoothed into a heart rate value for a time window based upon confidence and/or prior heart rate data.

### BACKGROUND

Heart rate is considered one of the more important and well-understood physiological measures. Researchers in a variety of fields have developed techniques that measure heart rate as accurately and unobtrusively as possible. These techniques enable heart rate measurements to be used by applications ranging from health sensing to games, along with interfaces that respond to a user's physical state.

One approach to measuring heart rate unobtrusively and inexpensively is based upon extracting pulse measurements from videos of faces, captured with an RGB (red, green, blue) camera. This approach found that intensity changes due to blood flow in the face was most apparent in the green video component channel, whereby this green component was used to extract estimates of pulse rate.

Existing video-based techniques are not robust, however. For example, the above technique based upon the green channel needs a very stable face image. Indeed, existing approaches (including those in deployed products) do not work well with even relatively slight levels of user movement and/or with variation in ambient lighting.

### SUMMARY

This Summary is provided to introduce a selection of representative concepts in a simplified form that are further described below in the Detailed Description. This Summary is not intended to identify key features or essential features of the claimed subject matter, nor is it intended to be used in any way that would limit the scope of the claimed subject matter.

Briefly, various aspects of the subject matter described herein are directed towards a video-based pulse measurement technology that in one or more aspects operates by computing pulse information from video signals of a subject captured by a camera over a time window. The technology includes processing signal data that contains the pulse information and that corresponds to at least one region of interest of the subject. The pulse information is extracted from the signal data, including by using motion data to reduce or eliminate effects of motion within the signal data. In one or more aspects, at least some of the motion data may be obtained from the video signals and/or from an external motion sensor.

One or more aspects include a signal quality estimator that is configured to receive candidate signals corresponding to a plurality of captured video signals of a subject. For each candidate signal, the signal quality estimator determines a signal quality value that is based at least in part upon the candidate signal's resemblance to pulse information. A heart rate extractor is configured to compute heart rate data corresponding to an estimated heart rate of the subject based at least in part upon the quality values.

One or more aspects are directed towards providing sets of feature data to a classifier, each set of feature data including feature data corresponding to video data of a subject captured at one of a plurality of regions of interest. Quality data is received from the classifier for each set of feature data, the quality data providing a measure of pulse information quality represented by the feature data. Pulse information is extracted from video signal data corresponding to the video data of the subject, including by using the quality data to select the video signal data. The feature data may include motion data as part of the feature data for each set.

Other advantages may become apparent from the following detailed description when taken in conjunction with the drawings.

### BRIEF DESCRIPTION OF THE DRAWINGS

The present invention is illustrated by way of example and not limited in the accompanying figures in which like reference numerals indicate similar elements and in which:

FIG. 1 is a block diagram illustrating example components that may be used in video based pulse measurement for heart rate detection, according to one or more example implementations.

FIG. 2 is a block diagram illustrating example components and data flow operations that may be used in video based pulse measurement for heart rate detection, according to one or more example implementations.

FIG. 3 is an example representation of region of interest detection and processing for a plurality of video-captured regions, according to one or more example implementations.

FIG. 4 is a block diagram showing example processing operations and example output at each such processing operation, according to one or more example implementations.

FIGS. 5A-5C are example representations of various aspects of motion filtering with respect to video-based pulse measurement, according to one or more example implementations.

FIGS. 6A-6C are example representations of feature extraction from signals showing normalized autocorrelation versus time for use in selecting signals for video-based pulse measurement, according to one or more example implementations.

FIG. 7A provides example representations of power spectra from selected components and corresponding values of peak confidence, according to one or more example implementations.

FIG. 7B is an example representation of waveforms in which classifier-provided confidence values are overridden by spectral peak confidence values with respect to selection, according to one or more example implementations.

FIGS. 8 and 9 comprise a flow diagram illustrating example steps that may be taken to determine heart rate from video signals according to one or more example implementations.

FIG. 10 is a block diagram representing an example non-limiting computing system or operating environment into which one or more aspects of various embodiments described herein can be implemented.

### DETAILED DESCRIPTION

Various aspects described herein are generally directed towards a robust video-based pulse measurement technology. The technology is based in part upon video signal quality estimation including one or more techniques for estimating the fidelity of a signal to obtain candidate signals. Further, given one or more signals that are candidates for extracting pulse and the quality estimation metrics, described are one or more techniques for extracting of heart rate from those signals in a more accurate and robust manner relative to prior approaches. For example, one technique compensates for motion of the subject based upon motion data sensed while the video is being captured.

Still further, temporal smoothing is described, such that given a series of heart rate values following extraction, (e.g., thirty seconds of heart rate values that were recomputed every second), described are ways of “smoothing” the heart rate signal/values into a measurement that is suitable for application-level use or presentation to a user. For example, data that indicate a heart rate that changes in a way that is not physiologically plausible may be discarded or otherwise have a lowered associated confidence.

It should be understood that any of the examples herein are non-limiting. For example, the technology is generally described in the context of heart rate estimation from video sources, however, alternative embodiments may apply the technology to other sources of heart rate signals. Such other source may include photoplethysmograms (PPGs, as used in finger pulse oximeters and heart-rate-sensing watches), electrocardiograms (ECGs), or pressure waveforms. Thus, the “candidate signals” referred to herein may include signals from one or more sensors (e.g., a red light sensor, a green light sensor, and a pressure sensor under a watch) or one or more locations (e.g., two different electrical sensors). A motion signal may be derived from an accelerometer in some situations, for example.

Further, while face tracking is one technique, another physiologically relevant region (or regions) of interest may be used. For example, the video signals or other sensor signals may be one or more patches of a subject's skin and/or eye.

As such, the present invention is not limited to any particular embodiments, aspects, concepts, structures, functionalities or examples described herein. Rather, any of the embodiments, aspects, concepts, structures, functionalities or examples described herein are non-limiting, and the present invention may be used various ways that provide benefits and advantages in heart rate estimation and signal processing in general.

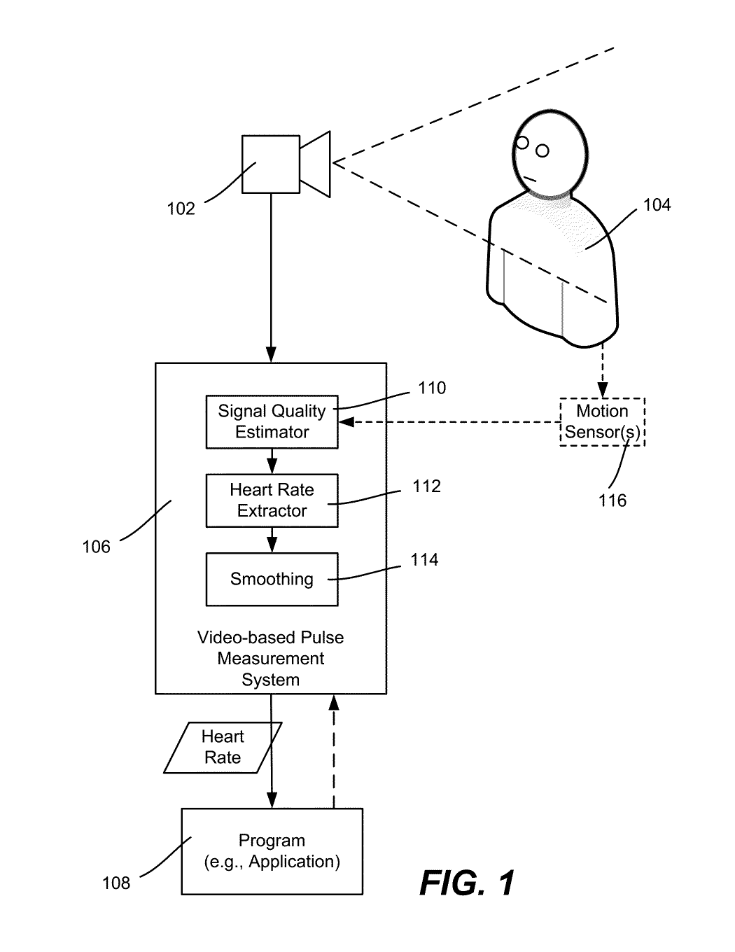

FIG. 1 is a block diagram showing one suitable implementation of the technology described herein. A camera 102 captures signals such as frames of RGB data of a human subject 104; other color schemes may be used, as may non-visible light frequencies such as infrared (IR). A video-based pulse measurement system 106 processes the received signal information and outputs suitable data, such as a current heart rate at regular intervals, to a program 108 such as an application, service or the like. For example, such an application may be running on a personal computer, smartphone, tablet computing device, handheld computing device, smart television, standalone device, exercise equipment, medical monitoring device and so on. Note that as indicated via the dashed arrow in FIG. 1, the program 108 may provide data to the video-based pulse measurement system 106, e.g., parameters such as a time window, quality and/or confidence thresholds, smoothing constraints, capabilities of the program, and so on. In this way, for example, an application in a piece of exercise equipment may operate in a different way than a game application that counts calories burned, for example.

Within the exemplified video-based pulse measurement system 106, a number of components may be present, such as generally arranged in a processing pipeline in one or more implementations. The components, which in this example include a signal quality estimator 110, a heart rate extractor 112 and a smoothing component 114, may be standalone modules, subsystems and so forth, or may be component parts of a larger program. Each of the components may include further components, e.g., the signal quality estimator 110 and/or the heart rate extractor 112 may include motion processing logic. Further, not all of the components may be present in a given implementation, e.g., smoothing need not be performed, or may be performed external to the video-based pulse measurement system 106. Additional details related to signal quality estimation, heart rate extraction and smoothing are provided below.

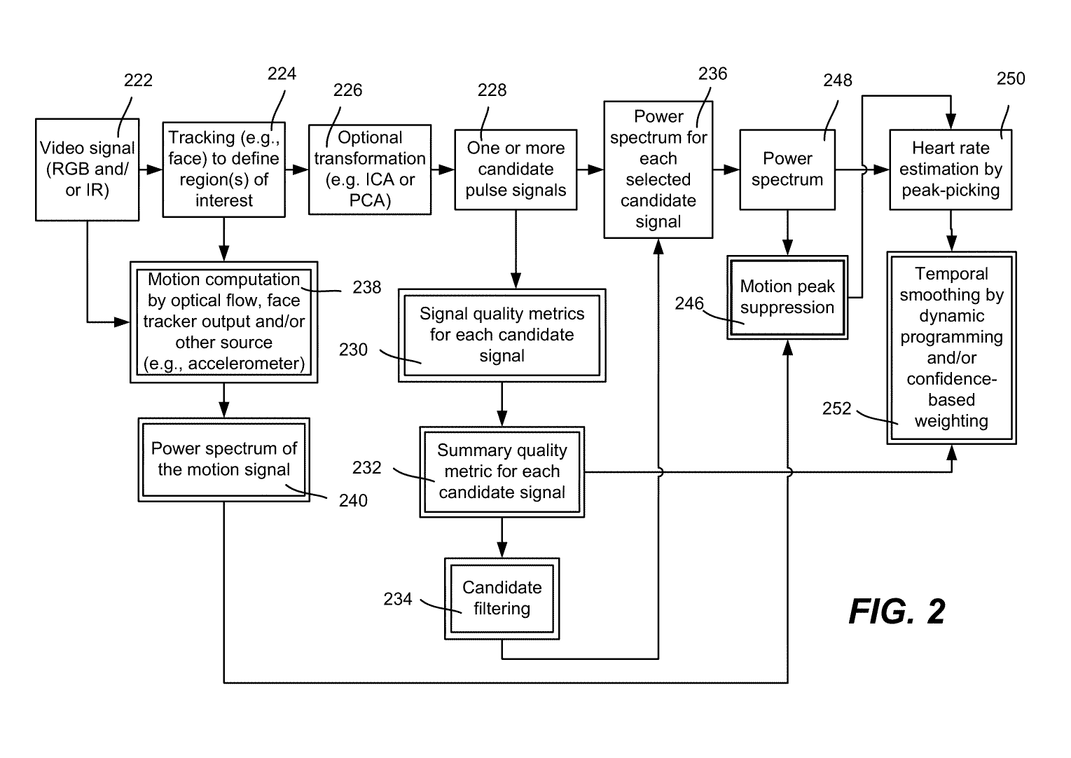

FIG. 2 is a general block diagram illustrating example components of one embodiment of a video-based pulse measurement system (such as the system 106 of FIG. 1). As is understood, the exemplified implementation of FIGS. 1 and 2 is based upon a combination of signal quality estimation, heart rate extraction and/or temporal smoothing.

In FIG. 2, an input video signal 222, which for example may contain RGB and/or infrared (IR) components, is provided to a face tracking mechanism 224. In general, the face tracking mechanism 224 locates and tracks one or more regions of interest, such as the face itself, the cheeks and so on. However as is understood, this is only one example, as any place other than the face where skin may be sensed (instead of or in addition to the face) may be selected as a region of interest, as may non-skin regions such as the eye or part of the eye. Note that known prior approaches sensed the whole face.

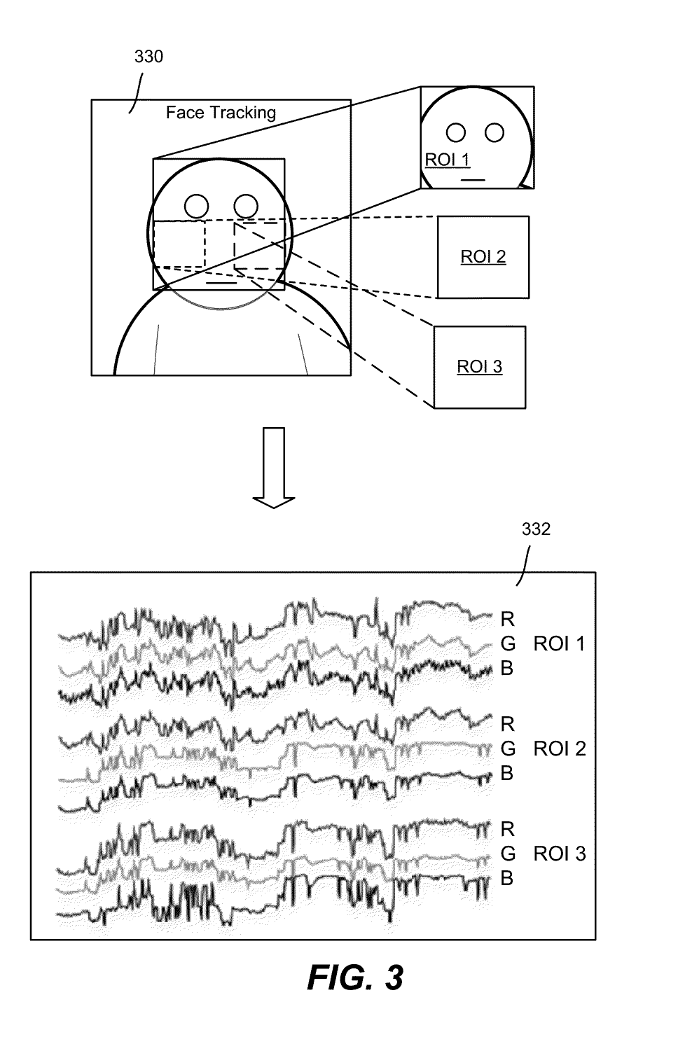

Region of interest tracking is generally exemplified as face tracking 330 in FIG. 3, in which regions of interest ROI 1, ROI 2 and ROI 3 provide R, G and B signals 332 for each region. In this example, a local average or the like may be computed from each ROI and each color channel, resulting in a total of nine intensity values (three regions by three component values) per frame. Note that this is only one example, and candidate signals need not be one-dimensional; for example, the technology/heuristics may be applied to the combined RGB signal instead of the individual RGB components. Note that it is feasible to use multiple cameras, which may be of the same type (e.g., RGB cameras) or a mix of camera types, (e.g., RGB and IR cameras)/

Conventional computer vision algorithms may be used to provide a face detector that yields approximate locations of the face (square) and the basic features (eyes, nose, and mouth) in each frame. However, in addition to the whole face (ROI 1), in the example of FIG. 3 the cheek regions are also extracted from each frame (ROIs 2 and 3). The cheeks tend to be useful because they are predominantly soft tissue that exhibit significant pulsatile changes with blood flow. This data may be band-pass smoothed e.g., with a second-order Butterworth filter with a pass band between 0.75 and 4 Hz, corresponding to 45-240 beats per minute. Note that the whole face may be considered a region of interest, and as shown in FIG. 3, regions of interest may overlap.

Returning to FIG. 2, the signals corresponding to the tracked regions may be transformed by a suitable transform 226 such as independent component analysis (ICA) or principal component analysis (PCA). This results in one or more candidate pulse signals 228.

The one or more candidate pulse signals 228 along with any related features may be processed (e.g., by a classifier/scorer) to obtain signal quality metrics 230 for each candidate signal, which may be combined or otherwise processed into summary quality metric data 232 for each candidate signal, as described below. Candidate filtering 234 may be used to select the top k (e.g., the top two) candidates based upon their quality values, which may be transformed into a power spectrum 236 for each candidate signal. As described herein, peak signals in the power spectrum 236 that may represent a pulse, but alternatively may be caused by motion of the subject, may be eliminated or at least lowered in quality estimation during heart rate estimation by the use of a similar motion power spectrum.

In general, the signal quality estimator 110 (FIG. 1) takes candidate signals that may contain information about pulse and determines the extent to which each candidate signal actually contains pulse information (providing a quality estimate). As one non-limiting example, a candidate signal may, for example, correspond to some number (e.g. thirty seconds) of data from just the green channel from a camera from a particular region of the image (e.g. the entire face, one cheek, and so forth, averaged down to one continuous signal. Two other non-limiting examples of candidate signals may be average values for some number (e.g. thirty seconds) of data from the red and blue channels, respectively. Still non-limiting examples are based upon some number (e.g. thirty seconds) of data from a transformation of the RGB signal from a region, e.g., the nine principal component vectors of the average RGB signals from three regions; each of the nine component vectors may be one candidate signal.

Signal quality estimation basically determines how much each of these candidate signals contains information about pulse. Various metrics or features may be used for estimating signal quality, and any number of such metrics may be put together into a classification or regression system to provide a unified measure of signal quality. Note that these metrics may be applied to each candidate signal separately.

In one or more implementations, the metrics are typically computed on windows of every candidate signal source, for example the last thirty seconds of the R, G, and B channels, recomputed every five seconds. However they may alternatively be run on an entire video or on very short segments of data.

Metrics for signal quality may include various features for signal quality from the autocorrelation of the signal. The autocorrelation is a standard transformation in signal processing that helps measure the repetitiveness of a signal. The autocorrelation of a one-dimensional signal produces another one-dimensional signal. The number of peaks in the autocorrelation and the magnitude of the first prominent peak in the autocorrelation are computed, (where “prominent” may be defined by a threshold height and a threshold distance from other peaks), along with the mean and variance of the spacing between peaks in the autocorrelation. Note that these are only examples of some useful autocorrelation-based features. Any number of heuristics related to repetitiveness that are derived from the autocorrelation may be used in addition to or instead of those described above.

Other features for signal quality may be derived, such as statistics on the time-domain signal itself, e.g. kurtosis, variance, number of zero crossings. Kurtosis is a useful time-domain statistic.

Still other features for signal quality may be derived by comparing the signal to a template of what known pulse signals look like, e.g. by cross-correlation or dynamic time warping. Pulse signals tend to have a characteristic shape that is not perfectly symmetric and does not look like typical random noise, and the presence or absence of this pattern may be exploited as a measure of quality. High correlation with a pulse template is generally indicative of high signal quality. This can be done using a static dictionary of pulse waveforms, or using a dynamic dictionary, e.g., populated from recent pulses observed in the current data stream that are assigned high confidence by other metrics.

Other features for signal quality may be derived from the power spectrum of the candidate signal. In particular, the power spectrum of a signal that represents heart rate tends to show a single peak around the heart rate. One implementation thus computes the magnitude ratio of the largest peak in the range of human heart rates to the second-largest peak, referred to as “spectral confidence.” If the largest peak is much larger than the next-largest-peak, this is indicative of high signal quality. The spectral entropy of the power spectrum, a standard metric used to describe the degree to which a spectrum is primarily concentrated around a single peak, may be similarly used for computing a spectral confidence value.

The following is a non-limiting set of signal data/feature data that may inform signal quality estimation, some or all of which may be fed into the classifier/scorer:
1) Motion information (from video or external, e.g., inertial sensors)
2) Light information from outside the ROI, either from other parts of the video signal and/or from a separate video/ambient light sensor
3) Previous observed heart rates
4) Distance between the camera and the user
5) Activity level (from motion, skeleton tracking, etc.)
6) Demographic information: height, weight, age, gender, race (particularly skin tone)
7) Temperature
8) Humidity
9) Other derived visual properties of the ROI, e.g. hairiness, sweatiness

Each of the metrics described herein may provide an independent estimate of how much a candidate signal contains information about pulse. To integrate these together into a single quality metric for a candidate signal, a supervised machine learning approach may be used, for example. In one example embodiment, these metrics are computed for every candidate signal in every thirty second window in a “training data set”, for which there is an external measure of the true heart rate (e.g., from an electrocardiogram). For each of those candidate signals, a human expert also may rate the candidate signal for its quality, and/or the signal is automatically rated by running a heart rate extraction process on the signal and comparing the result to the true heart rate. This is thus a very typical supervised machine learning problem, namely that a model is trained to take those metrics and predict signal quality given new data (for which the “true” heart rate is not known). The model may be continuous (producing an estimate of overall signal quality) or discrete (labeling the signal as “good” or “bad”). The model may be a simple linear regressor (as described in one example herein), or may be a more complex classifier/regressor (e.g. a boosted decision tree, neural network, and so forth).

With respect to heart rate estimation, given the candidate signals that may contain information about pulse, and the quality metrics for each signal, a next step in one embodiment is to determine the actual heart rate represented by some window of time, for which there may be multiple candidate heart rate signals. Another possible determination is that no heart rate can be extracted from this window of time.

Various techniques for extracting heart rate are described herein; note that these are not mutually exclusive. The exemplified techniques generally build on the basic approach of taking a Fourier (or wavelet) transform of a signal and finding the highest peak in the corresponding spectrum, within the range of frequencies corresponding to reasonable human heart rates.

Candidate filtering 234 is part of one method for estimating a heart rate, so as to choose one or more of the candidate signals for heart rate extraction. In one embodiment, candidate signals are ranked according to the quality score assigned in the prior phase, using a machine learning system to integrate the quality metrics into a single quality score for each candidate signal. Only the top k (e.g., the top two) signals, as ranked by the supervised classification system, are selected for further examination.

Given multiple possible peaks in the power spectrum 236 of a candidate signal that may correspond to heart rate, a conventional approach is to assume that the largest peak corresponds to heart rate. However, even if face tracking is used to define the region of interest so that in theory a moving face does not introduce motion artifact into the candidate heart rate signals, some amount of motion artifact virtually always remains in candidate signals. As a result, motion may remain a challenge for estimating heart rate from video streams. For example, even if a signal is pre-processed to minimize the effects of motion, some amount of motion is likely to remain in the candidate signals, and motion of a face is often very close in frequency to a human heart rate (about 1 Hz).

Thus, as described herein, motion may be estimated such as by a motion compensator 238 (computation mechanism) of FIG. 2 and used to suppress (e.g., eliminate or reduce the quality score of) heart rate signals that are likely to actually be motion-generated. More particularly, other features for signal quality may be derived by comparing the signal to an estimate of the motion pattern in the video from which these signals were derived, e.g. computed from the optical flow in the video stream or via face tracker output coordinates. Note however that motion signals may be sensed in many ways, including via an accelerometer, and any way or combination of ways of obtaining a reasonable motion power spectrum 240 may be used.

In general, if a candidate signal is very similar to the motion pattern (as computed by cross-correlation, for example), the candidate signal is statistically less likely to contain information about pulse, which may be used to lower its quality score as described herein. Such templates need not be only based on time, but also on space, as a true pulse signal does not appear uniformly across the face, as a pulse progresses across the face in a consistent pattern (which may vary from person to person) that relates to the density of blood vessels in different parts of the face and the orientation of the larger blood vessels delivering blood to the face. Consequently, a high correlation of the full space-time sequence of images with a known space-time template is indicative of high signal quality.

To obtain the motion power spectrum, the motion compensator 238 provides the motion power spectrum 240, which is generally used to assist in detecting when a person's coincidental movement may be causing the input video signal 222 to resemble a pulse. In other words, data (e.g., a transform) corresponding to the movement such as the power spectrum 240 of the motion signal may be used to lower the quality score (and thus potentially eliminate) one or more of the candidate signals 228 that look like quality pulse signals but are instead likely to be caused by the subject's motion. Note that the motion compensator 238 may be based upon determining motion from the video, and/or from one or more external motion sensors 116 (FIG. 1) such as an accelerometer.

In one implementation, the power spectrum of the motion signal may be used for motion peak suppressor (block 246), such as to a assign a lower weight to peaks in the power spectrum of the candidate heart rate signal that align closely with peaks in the power spectrum of the motion signal. That is, the system may pick a peak that is not the largest peak in the spectrum of the candidate signal, if that largest peak aligns too closely with probable motion frequencies.

Typically there are multiple candidate signals that were not filtered out in the filtering stage. Each remaining candidate signal has a power spectrum 248 that has been adjusted for similarity to the motion spectrum. To choose a final heart rate, one implementation uses a weighted combination of the overall quality estimate of each remaining candidate and the prominence of the peak that is believed to represent the heart rate in each of the chosen signals. Candidates with high signal quality and prominent heart rate peaks are preferred over candidates with lower signal quality and less prominent heart rate peaks, (where prominence is defined as a function of the distance to other peaks and the amplitude relative to adjacent valleys in the power spectrum 248).

At this stage, a candidate heart rate is selected, as shown via block 250 of FIG. 2. Using one or more of the quality metrics the system may decide that even the best heart rate signal is not of sufficient quality to report to an application or to a user, and this entire frame may be rejected, (e.g., the system outputs “heart rate not available” of the like). The quality metrics also may be provided to an application that is consuming the final heart rate signal, as applications may be interested in the quality metrics, for example to place more or less weight on a particular heart rate estimate when computing a user's caloric expenditure.

Temporal smoothing 252, such as based on the summary quality metric data 232, also may be used as described herein. For example, when an estimate of the current heart rate for a particular window in time is available, the estimates may vary significantly from one window to the next as a result of incorrect predictions. By way of example, a sequence of estimates separated by ten seconds each may be [70 bpm, 71 bpm, 140 bpm, 69 bpm] (where bpm is beats per minute). In this example, it is very likely that the estimate of 140 bpm was an error. As can be readily appreciated, reporting such rapid, unrealistic changes in heart rate that are likely errors is undesirable.

Described herein are example techniques for “smoothing” the series of heart rate estimates, including smoothing by dynamic programming and confidence-based weighting; note that these techniques are not mutually exclusive, and one or both may be used separately, together with one another, and/or with one or more other smoothing techniques.

With respect to smoothing by dynamic programming, the system likely still has multiple candidate peaks in the power spectrum that may represent heart rate (from multiple candidate signals and/or multiple peaks in each candidate signal's power spectrum). As described above, in one embodiment a single final heart rate estimate was chosen. As an alternative to choosing a single heart rate, a list or the like of the candidate heart rate values at each window in time may be maintained, with each value associated with a confidence score, (e.g., a combination of the signal quality metric for the candidate signal and the prominence of the peak itself in the power spectrum), with a dynamic programming approach used to select the “best series” of candidates across many windows in a sequence. The “best series” may be defined as the one that picks the heart rate values having the most confidence, subject to penalties for large, rapid jumps in heart rate that are not physiologically plausible.

With respect to confidence-based weighting, another approach to smoothing the series of heart rate measurements is to weight new estimates according to their confidence. A very high confidence score in a new estimate, possibly as high as one-hundred percent, may be used as a threshold for reporting that estimate right away. If there is more confidence in previous measurements than in the current measurement, the current and previous estimates may be blended according to the current confidence values and/or previous confidence values, for example as a linear (or other mathematical) combination weighted by confidence. Consider that the current heart rate estimate is h(t), the previous heart rate estimate is h(t−1), the current confidence value is α(t), and the previous confidence value is α(t−1). The following are some example schemes for confidence-based selection of the final reported heart rate h′(t).

Weight only according to current confidence:

h′(t)=α(t)h(t)+(1−α(t))h(t−1)

Weight according to current and previous confidences

$${h}^{′} ( t ) = \frac{α ( t )}{α ( t ) + α ( t - 1 )} h ( t ) + \frac{α ( t - 1 )}{α ( t ) + α ( t - 1 )} h ( t - 1 )$$

The above temporal smoothing is based upon using known physiological constraints (e.g., a heart rate can only change so fast) along with other factors related to signal quality, to more intelligently integrate across heart rate estimates that do not always agree. Such known physiological constraints can be dynamic, and can be informed by context. For example, a subject's heart rate is likely to change more rapidly when the subject is moving a lot, whereby information from a motion signal (coming from video and/or from an inertial sensor such as in a smartphone or watch) can inform the temporal smoothing method. For example, what is considered implausible for a person who is relatively still may not be considered implausible for a person who is rapidly changing motions.

The above technology has thus far been described in the context of heart rate estimation from video sources. However, alternative embodiments may apply these techniques to other sources of heart rate signals, such as photoplethysmograms (PPGs, as used in finger pulse oximeters and heart-rate-sensing watches), electrocardiograms (ECGs), or pressure waveforms. In these scenarios, the candidate signals may be signals from one or more sensors (e.g. a red light sensor, a green light sensor, and a pressure sensor under a watch) or one or more locations (e.g. two different electrical sensors). The motion signal may be derived from an accelerometer or other such inertial sensor in such cases, for example.

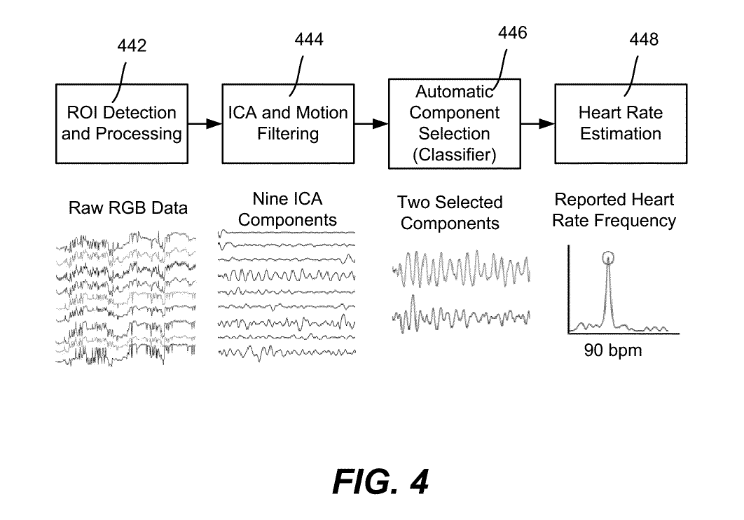

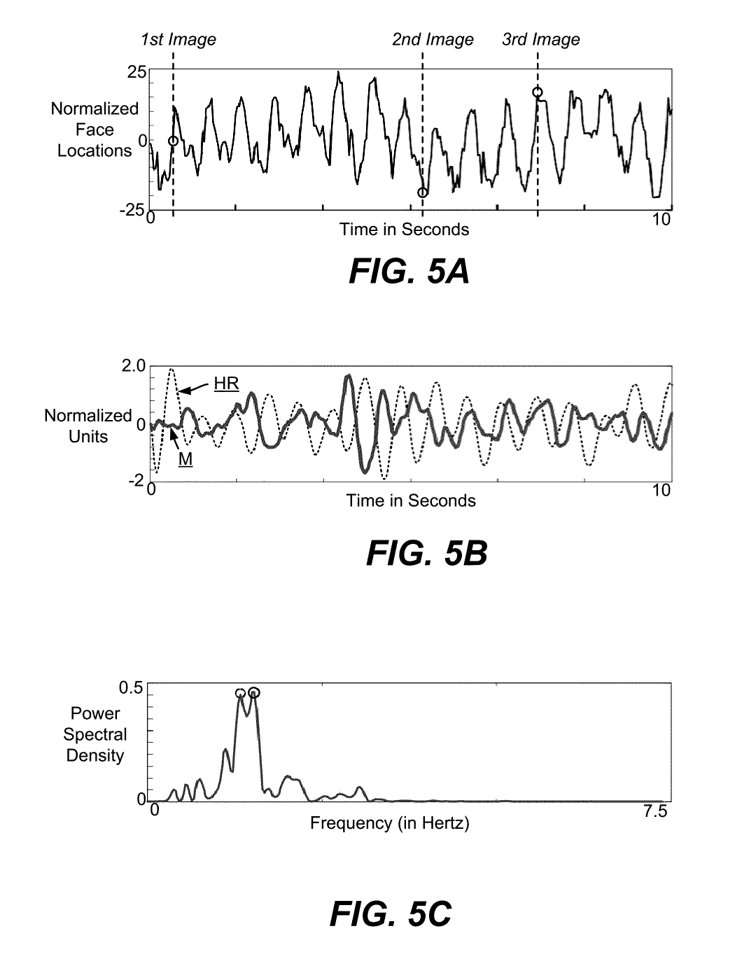

FIGS. 4 and 5 are directed towards additional details of an example implementation that achieves robust heart rate estimation through operations applied sequentially on video, (of regions of the face in this example). Such operations are shown in FIG. 4, and include region-of-interest detection and processing 442, signal separation and motion filtering 444, component selection 446 and heart rate estimation 448.

Micro-fluctuations due to blood flow in the face form temporally coherent sources due to their periodicity. A signal separation algorithm such as ICA is capable of separating the heart rate signal from other temporal noise such as intensity changes due to motion or environmental noise. In the exemplified implementation of FIG. 4, the red, green, and blue channels of the camera are treated as three separate sensors that record a mixture of signals originating from multiple sources.

ICA is well known for finding underlying factors from multi-variate statistical data, and may be more appropriate than methods like Principal Component Analysis (PCA). Notwithstanding, if a transformation is used, any suitable transformation may be used.

Applying region detection on N frames yielded an input data matrix X, of size 9×N, which can be represented as

X=AS (1)

where A is the matrix that contains weights indicating linear combination of multiple underlying sources contained in S. The S matrix of size 9×N contains the separated sources (called components), any one (or combination) of which may represent the signal associated with the pulse changes on the face. One implementation utilized the Joint Approximate Diagonalization of Eigenmatrices (JADE) algorithm to implement ICA. Note that forcing the number of output components to be equal to number of input mixed signals represents a dense model that helps separate unknown sources of noise with good accuracy.

With respect to motion filtering, natural head movements associated with daily activities such as watching television, performing desk work or exercising can significantly affect the accuracy of camera-based heart rate measurement. Longer periodic motions need to be considered; for example, changes in the position and intensity of specular and diffuse reflections on the face change while running or biking indoors as well as aperiodic motions, e.g., rapid head movements when switching gaze between multiple screens, to other objects in the environment or looking away from a screen.

Periodic motions cause large, temporally-varying color and intensity changes that are easily confused with variations due to pulse. This manifests itself as a highly correlated ICA component that captures motion-based intensity changes at multiple locations on the face. As facial motions often occur at rates in the same range of frequencies of heart rate, they cannot be ignored. An example is generally represented in FIGS. 5A-5C, which represent an example of motion filtering using large periodic motion. FIG. 5A shows three frames with different head positions and normalized head translation vectors derived from face tracking coordinates; FIG. 5B represents time domain signals for a selected heart rate signal (HR) and motion component (M) having a correlation with FIG. 5A equal to 0.89. FIG. 5C shows the power spectrum of the selected component with two peaks at heart rate and motion frequencies.

One or more implementations are directed toward solving the motion-related problems by tracking the head, in that that head motion may closely correlate with changes in the intensity of light reflected from the skin when a person's head is in motion. The 2-D coordinates indicating the face location (mean of top-left and bottom-right) may be used to derive an approximate value for head motion between subsequent frames (FIG. 5A). The total amount of head activity between two subsequent frames may be estimated using the partial derivative of the centroid of the face location with respect to frame number:

$$\begin{matrix}Δ {a}_{n} = \vert \frac{\partial }{δ n} ( \sqrt{{\overline{x}}_{n}^{2} + {\overline{y}}_{n}^{2}} ) \vert & ( 2 ) \\ a ( t ) = {\sum }_{n = 1}^{w} Δ {a}_{n} , & ( 3 )\end{matrix}$$

where α(t) represents the head activity within a window. One implementation empirically selected a window size w of 300 frames (10 seconds), as a smallest window feasible for heart rate detection. This metric may be used to automatically label each window as either motion or rest. A static threshold of twenty percent of the face dimension (length or width in pixels) was used for labeling windows. For example, if a face region is 200×200 pixels, the motion threshold for a ten-second window is set to 400 (0.2×200 pixels×10 sec). If the total head translation α(t) is greater than 400 pixels (over the 10 second window), the window is labeled as motion. These labels guide the processing and assist in heart rate estimation. For example, the heart rate is expected to be higher during periods of exercise (motion) than during rest periods.

By way of example, motion filtering us generally represented in FIGS. 5A-5C using an example with large periodic motion. FIG. 5A shows three frames with different head positions and normalized head translation vectors derived from face tracking coordinates. FIGS. 5B and 5C show time domain signals for the selected signal and motion component, having correlation=0.89 with FIG. 5A, and the power spectrum of the selected component with two peaks at heart rate (HR) and motion (M) frequencies.

In this example, FIG. 5A illustrates approximate head motion values with the threshold set at 380 (face size 190×190 pixels), while a user alternates between blocks of cycling on an exercise bike and sitting still. The heart rate is expected to be higher during periods of exercise (motion) than the rest periods as illustrated in FIGS. 5B and 5C by corresponding heart rate (HR) estimates from the camera and the optical sensor. The heart rate drops rapidly at the end of each biking cycle as the user comes to a rest.

If the window is labeled as motion, any periodic signals related to the motion may be ignored by removing them. To do this, the component matrix S may be cross-correlated with the normalized face locations (Equation (2)) for that window.

To remove components that dominantly represent head motion, the rows in the component matrix S with a correlation greater than 0.5 (e.g., empirically determined) are discarded from further calculations. This motion filtering results in matrix S′. A global threshold for subjects can consistently reject components associated large motion artifacts. If the window is given a rest label, no components are removed and the computation proceeds to the next stage, shown in FIG. 4 as automatic component selection 446.

Periodic head motion may be visually and statistically similar to one of the nine components derived from the raw data. The statistical similarity may confuse a peak detection method that relies on a MAP-estimate, causing it to falsely report the highest peak in the power spectrum as heart rate. Thus, prior knowledge of the head motion frequency assists in picking the correct heart rate, even if the signal is largely dominated by head-motion-induced changes. Certain common types of aperiodic movements also may occur, such as induced when individuals scratch their face or turn their head, or perform short-duration body movements.

Component identification benefits from this preprocessing step as it enables unsupervised selection of the heart rate component and eliminates uncertainty associated with the arbitrary component ordering, which is a fundamental property of ICA methods.

With respect to component selection 446 in the exemplified implementation of FIG. 4, heart rate component identification may be treated as a classification and detection problem that can be divided into feature extraction and classification Feature extraction derives a number of features primarily associated with the regularity of the signal, in that the underlying morphology (and dominant frequency) of a pulse waveform can be characterized by the number of regularly-spaced peaks. This is followed by classification, where a linear classifier or the like may be employed to estimate each candidate component's likelihood to be a pulse wave. The top two components (chosen for a variety of reasons set forth herein) are utilized for peak detection and heart rate estimation.

With respect to feature extraction, the component classification system makes use of a number of features (nine in this example) generally derived using the autocorrelation of each component. The autocorrelation value at a time instant t represents the correlation of the signal with a shifted version of itself (shifted by t seconds). Because the pulse waveform is reasonably periodic, autocorrelation effectively differentiate these waveforms from noise.

If a signal has dominant periodic trend (of period T), the autocorrelation has high magnitude at shift T. The process computes the autocorrelation of each candidate component in matrix S′, and normalizes the autocorrelation signal so the value at a shift of zero is one. For each of these nine auto-correlations (one for each component), a number of features (e.g., eight in this example) that were observed as the most valuable indicators of regularity are computed.

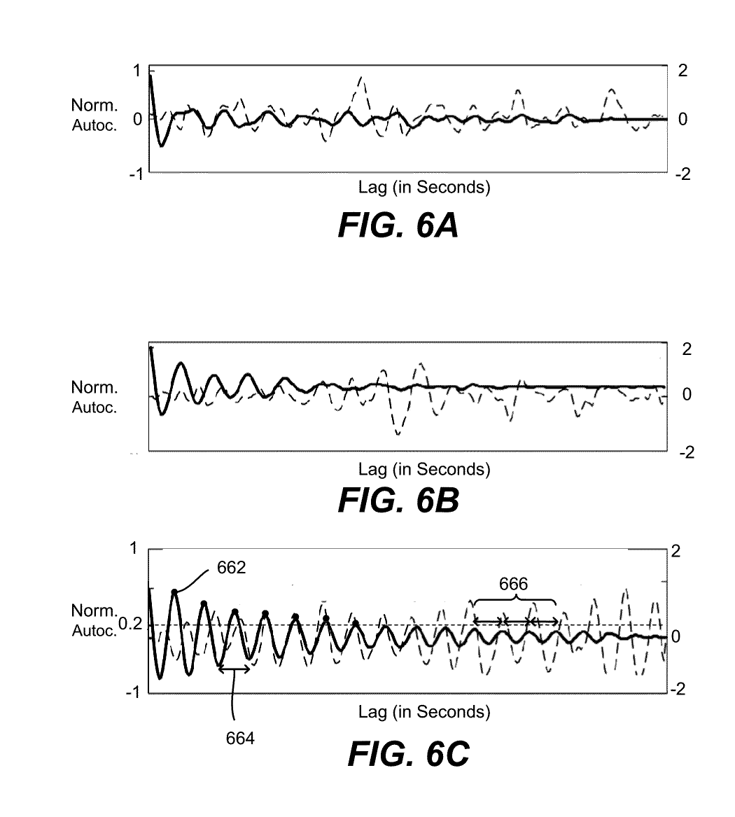

A first feature is the total number of “prominent” peaks, such as the number of peaks greater than a static threshold (e.g., 0.2, set based on preliminary experiments) and located at least a threshold shift away from the neighboring peaks (0.33 seconds). FIGS. 6A-6C represent some of the feature extraction concepts; FIG. 6A shows a noise component, FIG. 6B an ambiguous component, and FIG. 6C a true heart rate waveform.

More particularly, FIGS. 6A-6C represent feature properties for data within a single time window selected from training data. The autocorrelation waveforms (solid lines) from the three selected components (dashed lines) each represent different autocorrelation properties/characteristics of the selected features that are used by the classifier.

The autocorrelation in FIG. 6C is labeled to highlight some of the features used by the classifier to label this component as heart rate. In the example of FIG. 6C, it is seen that the magnitude of the first peak 662 is greater than or equal to 0.2, and that the number of “best” peaks (greater than or equal to 0.2, represented by a dot at the top of each such peak) is seven. In this example, the minimum peak-to-peak lag, represented by arrow 664, is greater than or equal to 0.33 seconds. The mean and variance of the peak-to-peak lags are represented via the arrows labeled 666. The threshold for minimum spacing (FIG. 6A-6C) may be chosen based on the maximum reasonable heart rate for a healthy user (e.g., 180 beats per minute). Note that peaks occurring closer than the threshold may not be characteristic of a regular pulse waveform.

A second feature is the magnitude of the first “prominent” peak, excluding the initial peak, at zero lag, which is always equal to one. Periodic signals yield a higher value for this feature (FIG. 6C).

A third feature is computed as the product of the first two features, and helps resolve ambiguous cases where the highest peaks in two different candidate components have equal magnitude and lag (see e.g., FIG. 6B versus FIG. 6C).

Other features include the mean and variance of peak-to-peak spacing (another measure of the periodicity of the signal), log entropy of the power spectrum of the autocorrelation (high entropy suggests multiple dominant frequencies), the first prominent peak's lag, and the total number of positive peaks.

Another feature, not derived from the autocorrelation, is the kurtosis of the time-domain component signal. This is primarily a measure of how non-Gaussian the signal is in terms of its probability distribution, that is, the “peaky-ness” of a discrete signal, similar to some of the autocorrelation features. The kurtosis values of each component in S′ are combined with the eight autocorrelation features in this example to provide the nine features.

Turning to classification, to determine which component out of the nine estimated components is most likely to contain the heart rate estimate, a classifier may be used, e.g., a linear classifier (regression model). The training data comprised ten-second sliding windows (one-second step) with nine candidate components estimated in each window. The training labels (binary) were assigned in a supervised manner by comparing the ground truth heart rate (optical pulse sensor waveform) with each component. Any component where the highest power spectrum peak was located within ±2 beats per minute (bpm) of the actual heart rate was assigned a positive label.

For each window in the test datasets, the feature matrix (of size nine features by nine components) is estimated and used with the classifier to obtain a binary label and a posteriori decision value a for each component. A signal-quality-driven peak detection approach, described herein, is applied to the best two components (the two highest a values) to estimate heart rate.

For heart rate estimation, the classifier provides confidence values for each ICA component to narrow in on the candidate component most likely to contain the pulse signal. Typically, multiple components are classified as likely heart rate candidates due to their heart rate-like autocorrelation feature values; this is particularly true with periodic motion, such as during exercise (even after motion filtering). In this example implementation, the process uses two signal quality metrics that reduce ambiguity in picking the frequency that corresponds to heart rate. In general, after applying such metrics in this example as described below, the highest peak in the power spectrum of the component selected by the metrics is reported as the estimated heart rate, h(t).

A first metric is the confidence value a provided by the classifier. The nine components are sorted based on this value with the highest k (e.g., two) chosen for further processing in the frequency domain.

A second metric is based on the power spectrum of each selected component. For each of these k components, the process estimates the power spectrum obtains the highest two peak locations and their magnitudes (within the window of 0.75-3 Hz, corresponding to 45-180 bpm). The peak magnitudes n₁ and n₂ are further used to estimate the spectral peak confidence (β) for each component as β₁=1−n₂/n₁ where i denotes the sorted component index (1 or 2, with α₁≧α₂) and peak magnitudes n₁≧n₂.

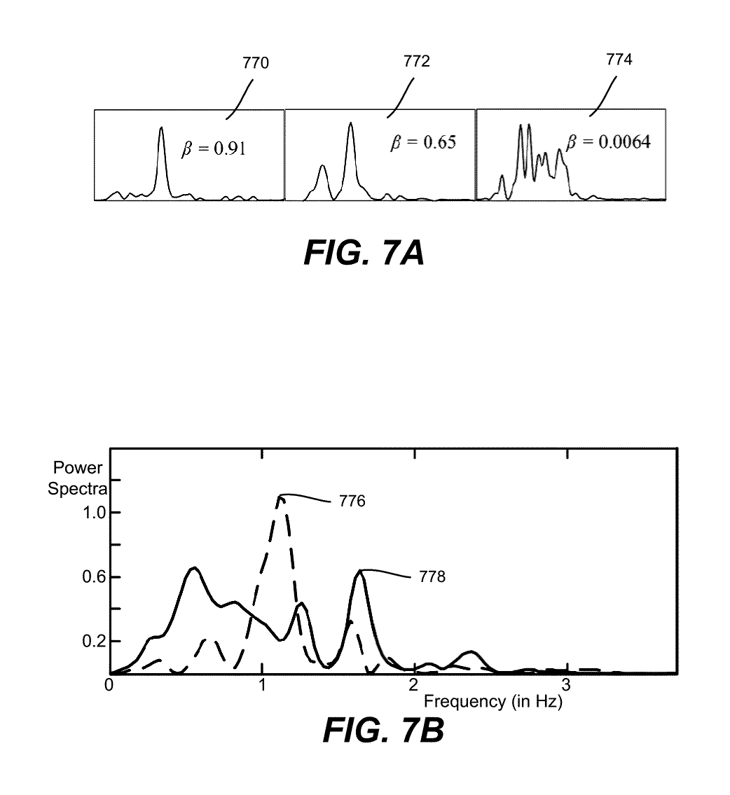

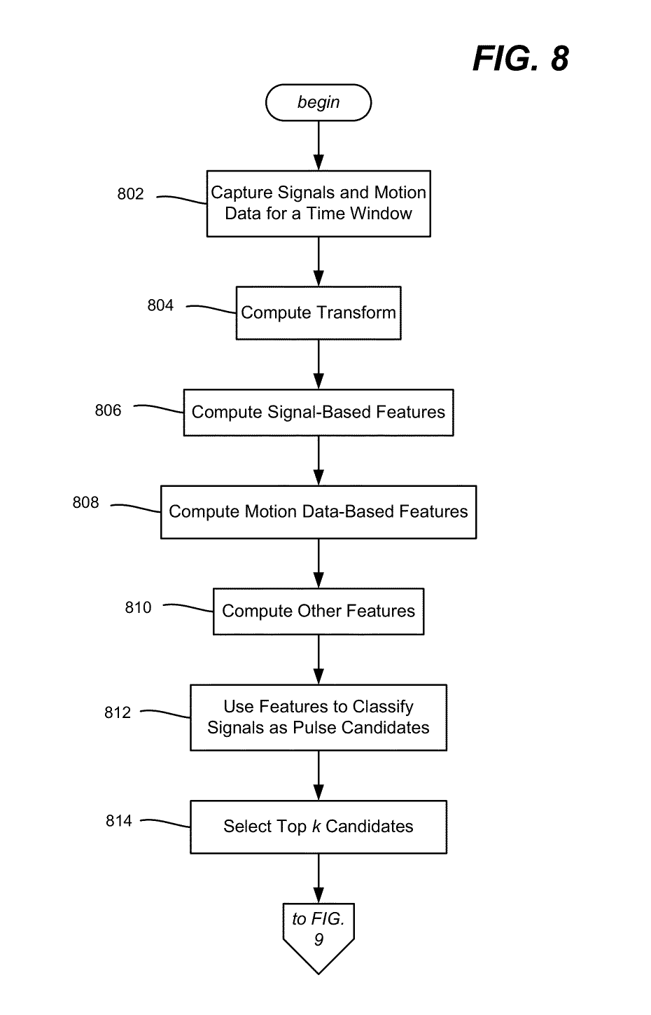

Spectral peak confidence is a good measure of the fitness of the component. FIG. 7A shows examples of power spectra from example components that illustrate a wide range of corresponding values of peak confidence β. As shown in the examples labeled 770, 772 and 744, the larger the differences of the peaks' magnitudes, the closer β is to one (1), e.g., example 770, whereas nearly equal magnitudes force β closer to zero, e.g., example 774) The peak confidences may be sorted to determine the index that is more likely to contain a clean peak signal. Note that this metric is not necessary when a single candidate component is labeled by the classifier, in which case the highest peak for this component is reported.

FIG. 7B shows an example where α₁=0.83≧α₂=0.75 (as determined by the classifier), but the second heart rate component is selected over the first component based on β₂=0.82≧β₁=0.19, that is, the β metric disagrees with the classifier output. A reason for developing a peak quality metric such as β is to avoid detection errors due to low-frequency noise. In FIG. 7B, the actual component (the dashed line with peak 776 (α₂=0.75, β₂=0.82)) is labeled by the classifier as the second-best component relative to other component (the solid line with peak 778 (α₁=0.83, β₁=0.19)), which may result in a poor heart rate estimate without the application of the peak confidence β. In practice this metric is useful in cases where the proposed motion filtering approach was unable to completely remove the noise due to periodic intensity changes. Note that it is alternatively feasible to include β as a feature for the classifier.

In this particular example, determining the final heart rate comprises a confidence-based weighting. In a real world scenario, there are multiple sources of noise (short and/or long duration), other than exercise-type motion that may corrupt the signal due to large intensity changes. Some of these may include camera noise, flickering lights, talking, head-nodding, laughing, yawning, observing the environment, and face-occluding gestures. To address such noise, the decision value a (from the classifier) may be used as a signal quality index to weight the current heart rate estimate before reporting it. For example, the final reported heart rate value h′(t) may be estimated using the previous heart rate h(t−1) and the current estimated heart rate h(t):

h′(t)=αh(t)+(1−α)h(t−1). (4)

The weighting presented here assists in minimizing large errors when the decision values are not high enough to indicate excellent signal quality. This model also plays a role in keeping track of the most recent stable heart rate in a continuous-monitoring scenario with or without motion artifacts. Note that performance of such a prediction model is largely dependent on the current window's estimate and the weight. At the end of this example process, a final heart rate h′(t) is computed for each ten second overlapping window in a video sequence.

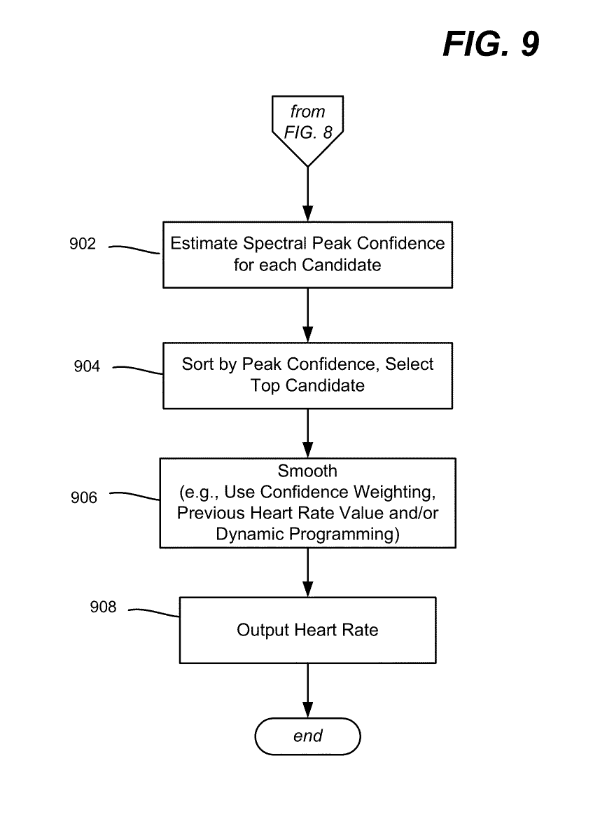

FIGS. 8 and 9 comprise a flow diagram summarizing various aspects of the technology described herein, beginning at step 802 which represents capturing signals and motion data for a time window. The signals may be obtained from a plurality of regions of interest. As is understood, the steps of FIGS. 8 and 9 may be repeated for each time window.

Step 804 represents computing the ICA or other transform from the signals. Step 806 processes the (e.g., transformed) signal data into the signal-based features described above.

Step 808 represents computing the motion data-based features. Note that this is used in alternatives in which the classifier is trained with motion data. It is alternatively feasible to use the motion data in other ways, e.g., to remove peak signals or lower confidence scores of peak signals based upon alignment with motion data, and so on.

Step 810 represents computing any other features that may be used in classification. These may include some or all of the (non-limiting) examples enumerated above, e.g., light information, distance data, activity level, demographic information, environmental data (temperature, humidity), visual properties and so on.

Step 812 feeds the computed feature data into the classifier, which in turn classifies the signals with respect to their quality as pulse candidates, e.g., each with a confidence score. The top k (e.g., two) candidates are selected from the classifier provided confidence scores at step 814. The exemplified steps continue in FIG. 9.

Step 902 of FIG. 9 represents estimating the spectral peak confidence for each candidate, e.g., the β value computed based upon the magnitudes of the two highest peaks. Step 904 represents sorting the top k candidates by their peak confidence values.

Step 906 represents the smoothing operation. As described above, this may be based upon the previous value and the confidence score of the current value (e.g., equation (4)), and/or via another smoothing technique such as dynamic programming. Step 908 outputs the heart rate as modified by any smoothing in this example.

As can be seen, there is described a technology in which video-based heart rate measurements are more accurate and robust than previous techniques, including via sensing multiple regions of interest, motion filtering and/or automatic component selection to identify and process candidate waveforms for pulse estimation. Classification may be used to provide top candidates, which may be combined with other confidence metrics and/or temporal smoothing to produce a final heart rate per time window.

One or more aspects are directed towards computing pulse information from video signals of a subject captured by a camera over a time window, including processing signal data that contains the pulse information and that corresponds to at least one region of interest of the subject. The pulse information is extracted from the signal data, including by using motion data to reduce or eliminate effects of motion within the signal data. In one or more aspects, at least some of the motion data may be obtained from the video signals and/or from an external motion sensor.

Processing the signal data may comprise inputting the signal data and the motion data into a classifier, and receiving a signal quality estimation from the classifier. The signal quality estimation may be used to determine one or more candidate signals for extracting the pulse information. Processing the signal data may comprise processing a plurality of signals corresponding to a plurality of regions of interest and/or corresponding to a plurality of component signals. Processing the signal data may comprise performing a transformation on the video signals.

Heart rate data may be computed from the pulse information, and used to output a heart rate value based upon the heart rate data. This may include smoothing the heart rate data into the heart rate value based at least in part upon prior heart rate data, a confidence score, and/or dynamic programming.

One or more aspects include a signal quality estimator that is configured to receive candidate signals corresponding to a plurality of captured video signals of a subject. For each candidate signal, the signal quality estimator determines a signal quality value that is based at least in part upon the candidate signal's resemblance to pulse information. A heart rate extractor is configured to compute heart rate data corresponding to an estimated heart rate of the subject based at least in part upon the quality values.

A transform may be used to transform the captured video signals into the candidate signals. A motion suppressor may be coupled to or incorporated into the signal quality estimator, including to modify any candidate signal that is likely affected by motion based upon motion data sensed from the video signals and/or sensed by one or more external sensors.

The signal quality estimator may incorporate or be coupled to a machine-learned classifier, in which signal feature data corresponding to the candidate signals is provided to the classifier to obtain the quality values. Other feature data provided to the classifier may include motion data, light information, previous heart rate data, distance data, activity data, demographic information, environmental data, and/or data based upon visual properties.

The heart rate extractor may compute the data corresponding to a heart rate of the subject by selection of a number of selected candidate signals according to the quality values, and by choosing one of the selected candidate signals as representing pulse information based upon relationships of at least two peaks within each of the selected candidate signals. A heart rate smoothing component may be coupled to or incorporated into the heart rate extractor to smooth the heart rate data into a heart rate value based upon confidence data and/or prior heart rate data.

One or more aspects are directed towards providing sets of feature data to a classifier, each set of feature data including feature data corresponding to video data of a subject captured at one of a plurality of regions of interest. Quality data is received from the classifier for each set of feature data, the quality data providing a measure of pulse information quality represented by the feature data. Pulse information is extracted from video signal data corresponding to the video data of the subject, including by using the quality data to select the video signal data. Providing the sets of feature data to the classifier may include providing motion data as part of the feature data for each set. Heart rate data may be computed from the pulse information, to output a heart rate value based upon the heart rate data.

### Example Operating Environment

It can be readily appreciated that the above-described implementation and its alternatives may be implemented on any suitable computing device or similar machine logic, including a gaming system, personal computer, tablet, DVR, set-top box, smartphone, standalone device and/or the like. Combinations of such devices are also feasible when multiple such devices are linked together. For purposes of description, a gaming (including media) system is described as one example operating environment hereinafter. However, it is understood that any or all of the components or the like described herein may be implemented in storage devices as executable code, and/or in hardware/hardware logic, whether local in one or more closely coupled devices or remote (e.g., in the cloud), or a combination of local and remote components, and so on.

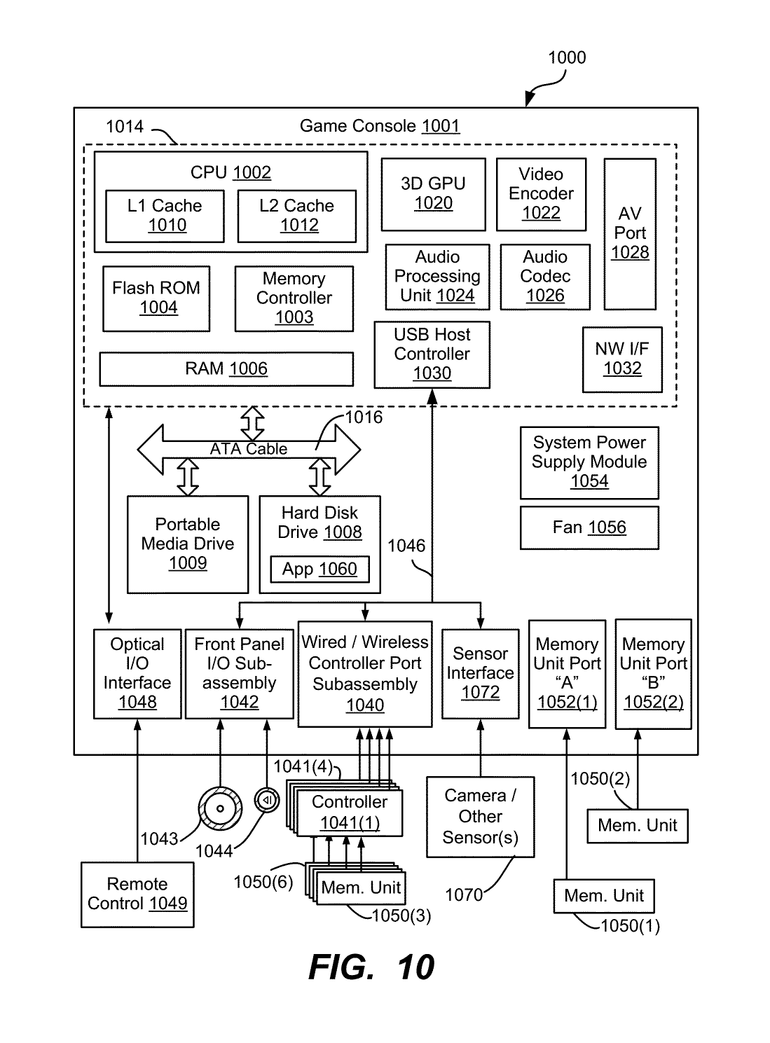

FIG. 10 is a functional block diagram of an example gaming and media system 1000 and shows functional components in more detail. Console 1001 has a central processing unit (CPU) 1002, and a memory controller 1003 that facilitates processor access to various types of memory, including a flash Read Only Memory (ROM) 1004, a Random Access Memory (RAM) 1006, a hard disk drive 1008, and portable media drive 1009. In one implementation, the CPU 1002 includes a level 1 cache 1010, and a level 2 cache 1012 to temporarily store data and hence reduce the number of memory access cycles made to the hard drive, thereby improving processing speed and throughput.

The CPU 1002, the memory controller 1003, and various memory devices are interconnected via one or more buses (not shown). The details of the bus that is used in this implementation are not particularly relevant to understanding the subject matter of interest being discussed herein. However, it will be understood that such a bus may include one or more of serial and parallel buses, a memory bus, a peripheral bus, and a processor or local bus, using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus.

In one implementation, the CPU 1002, the memory controller 1003, the ROM 1004, and the RAM 1006 are integrated onto a common module 1014. In this implementation, the ROM 1004 is configured as a flash ROM that is connected to the memory controller 1003 via a Peripheral Component Interconnect (PCI) bus or the like and a ROM bus or the like (neither of which are shown). The RAM 1006 may be configured as multiple Double Data Rate Synchronous Dynamic RAM (DDR SDRAM) modules that are independently controlled by the memory controller 1003 via separate buses (not shown). The hard disk drive 1008 and the portable media drive 1009 are shown connected to the memory controller 1003 via the PCI bus and an AT Attachment (ATA) bus 1016. However, in other implementations, dedicated data bus structures of different types can also be applied in the alternative.

A three-dimensional graphics processing unit 1020 and a video encoder 1022 form a video processing pipeline for high speed and high resolution (e.g., High Definition) graphics processing. Data are carried from the graphics processing unit 1020 to the video encoder 1022 via a digital video bus (not shown). An audio processing unit 1024 and an audio codec (coder/decoder) 1026 form a corresponding audio processing pipeline for multi-channel audio processing of various digital audio formats. Audio data are carried between the audio processing unit 1024 and the audio codec 1026 via a communication link (not shown). The video and audio processing pipelines output data to an A/V (audio/video) port 1028 for transmission to a television or other display/speakers. In the illustrated implementation, the video and audio processing components 1020, 1022, 1024, 1026 and 1028 are mounted on the module 1014.

FIG. 10 shows the module 1014 including a USB host controller 1030 and a network interface (NW I/F) 1032, which may include wired and/or wireless components. The USB host controller 1030 is shown in communication with the CPU 1002 and the memory controller 1003 via a bus (e.g., PCI bus) and serves as host for peripheral controllers 1034. The network interface 1032 provides access to a network (e.g., Internet, home network, etc.) and may be any of a wide variety of various wire or wireless interface components including an Ethernet card or interface module, a modem, a Bluetooth module, a cable modem, and the like.

In the example implementation depicted in FIG. 10, the console 1001 includes a controller support subassembly 1040, for supporting at least four game controllers 1041(1)-1041(4). The controller support subassembly 1040 includes any hardware and software components needed to support wired and/or wireless operation with an external control device, such as for example, a media and game controller. A front panel I/O subassembly 1042 supports the multiple functionalities of a power button 1043, an eject button 1044, as well as any other buttons and any LEDs (light emitting diodes) or other indicators exposed on the outer surface of the console 1001. The subassemblies 1040 and 1042 are in communication with the module 1014 via one or more cable assemblies 1046 or the like. In other implementations, the console 1001 can include additional controller subassemblies. The illustrated implementation also shows an optical I/O interface 1048 that is configured to send and receive signals (e.g., from a remote control 1049) that can be communicated to the module 1014.

Memory units (MUs) 1050(1) and 1050(2) are illustrated as being connectable to MU ports “A” 1052(1) and “B” 1052(2), respectively. Each MU 1050 offers additional storage on which games, game parameters, and other data may be stored. In some implementations, the other data can include one or more of a digital game component, an executable gaming application, an instruction set for expanding a gaming application, and a media file. When inserted into the console 1001, each MU 1050 can be accessed by the memory controller 1003.

A system power supply module 1054 provides power to the components of the gaming system 1000. A fan 1056 cools the circuitry within the console 1001.

An application 1060 comprising machine instructions is typically stored on the hard disk drive 1008. When the console 1001 is powered on, various portions of the application 1060 are loaded into the RAM 1006, and/or the caches 1010 and 1012, for execution on the CPU 1002. In general, the application 1060 can include one or more program modules for performing various display functions, such as controlling dialog screens for presentation on a display (e.g., high definition monitor), controlling transactions based on user inputs and controlling data transmission and reception between the console 1001 and externally connected devices.

As represented via block 1070, a camera (including visible, IR and/or depth cameras) and/or other sensors, such as a microphone, external motion sensor and so forth may be coupled to the system 1000 via a suitable interface 1072. As shown in FIG. 10, this may be via a USB connection or the like, however it is understood that at least some of these kinds of sensors may be built into the system 1000.

The gaming system 1000 may be operated as a standalone system by connecting the system to high definition monitor, a television, a video projector, or other display device. In this standalone mode, the gaming system 1000 enables one or more players to play games, or enjoy digital media, e.g., by watching movies, or listening to music. However, with the integration of broadband connectivity made available through the network interface 1032, gaming system 1000 may further be operated as a participating component in a larger network gaming community or system.

### CONCLUSION

While the invention is susceptible to various modifications and alternative constructions, certain illustrated embodiments thereof are shown in the drawings and have been described above in detail. It should be understood, however, that there is no intention to limit the invention to the specific forms disclosed, but on the contrary, the intention is to cover all modifications, alternative constructions, and equivalents falling within the spirit and scope of the invention.

What is claimed is:

1. A method comprising, computing pulse information from video signals of a subject captured by a camera over a time window, including processing signal data that contains the pulse information and that corresponds to at least one region of interest of the subject, and extracting the pulse information from the signal data, including using motion data to reduce or eliminate effects of motion within the signal data.

2. The method of claim 1 wherein processing the signal data comprises inputting the signal data and the motion data into a classifier, and further comprising, receiving a signal quality estimation from the classifier, and using the signal quality estimation to determine one or more candidate signals for extracting the pulse information.

3. The method of claim 1 wherein processing the signal data comprises processing a plurality of signals corresponding to a plurality of regions of interest, or processing a plurality of signals corresponding to a plurality of component signals, or both processing a plurality of signals corresponding to a plurality of regions of interest and processing a plurality of signals corresponding to a plurality of component signals.

4. The method of claim 1 wherein extracting the pulse information from the signal data comprises extracting feature data.

5. The method of claim 1 wherein extracting the feature data comprises determining feature data corresponding to at least one of: autocorrelation data, spectral entropy data, motion data, light information, previous heart rate data, distance data, activity data, demographic information, environmental data, or data based upon visual properties.

6. The method of claim 1 further comprising, obtaining at least some of the motion data from the video signals.

7. The method of claim 1 further comprising, obtaining at least some of the motion data from an external motion sensor.

8. The method of claim 1 wherein the pulse information corresponds to heart rate data, and further comprising, smoothing the heart rate data based at least in part upon prior heart rate data.

9. The method of claim 1 wherein the pulse information corresponds to heart rate data, and further comprising, smoothing the heart rate data based at least in part upon a confidence score.

10. The method of claim 1 wherein the pulse information corresponds to heart rate data, and further comprising, smoothing the heart rate data based at least in part upon dynamic programming.

11. A system comprising:
a signal quality estimator, the signal quality estimator configured to receive candidate signals corresponding to a plurality of captured video signals of a subject, and for each candidate signal, the signal quality estimator further configured to determine a signal quality value that is based at least in part upon feature data extracted from candidate signal, and
a heart rate extractor, the heart rate extractor configured to compute heart rate data corresponding to an estimated heart rate of the subject based at least in part upon the quality values.

12. The system of claim 11 further comprising a motion suppressor coupled to or incorporated into the signal quality estimator, the motion suppressor configured to modify any candidate signal that is likely affected by motion based upon motion data sensed from the video signals or sensed by one or more external sensors, or both sensed from the video signals and sensed by one or more external sensors.

13. The system of claim 11 wherein the feature data correspond to spectral entropy data or autocorrelation data, or both.

14. The system of claim 11 wherein the heart rate extractor is configured to compute the data corresponding to a heart rate of the subject by selection of a number of selected candidate signals according to the quality values, and to choose one of the selected candidate signals as representing pulse information based upon relationships of at least two peaks within the power spectrum of each of the selected candidate signals.

15. The system of claim 11 wherein the signal quality estimator incorporates or is coupled to a machine-learned classifier, in which signal feature data corresponding to the candidate signals is provided to the classifier to obtain the quality values.

16. The system of claim 15 further comprising other feature data provided to the classifier, including feature data corresponding to at least one of: motion data, light information, previous heart rate data, distance data, activity data, demographic information, environmental data, or data based upon visual properties.

17. The system of claim 11 further comprising a heart rate smoothing component coupled to or incorporated into the heart rate extractor, the heart rate smoothing component configured to smooth the heart rate data into a heart rate value based upon confidence data or prior heart rate data, or based upon both confidence data and prior heart rate data.

18. One or more machine-readable storage devices or machine logic having executable instructions, which when executed perform steps, comprising:
providing sets of feature data to a classifier, each set of feature data including feature data corresponding to video data of a subject captured at one of a plurality of regions of interest;
receiving quality data from the classifier for each set of feature data, the quality data for each set of feature data providing a measure of pulse information quality represented by the feature data; and
extracting pulse information from video signal data corresponding to the video data of the subject, including using the quality data to select the video signal data.

19. The one or more machine-readable storage devices or machine logic of claim 18 wherein providing the sets of feature data to the classifier comprises providing motion data as part of the feature data for each set.

20. The one or more machine-readable storage devices or machine logic of claim 18 having further executable instructions comprising computing heart rate data from the pulse information, and outputting a heart rate value based upon the heart rate data.
